(19) United States
(12) Patent Application Publication
(10) Pub. No.: US 2017/0333450 A1
(43) Pub. Date: Nov. 23, 2017

(54) COMPOSITIONS AND METHODS FOR TREATING HIV-ASSOCIATED COGNITIVE DYSFUNCTION

(71) Applicants: The Regents of the University of California, Oakland, CA (US); The Trustees of the University of Pennsylvania, Philadelphia, PA (US)

(72) Inventors: Andrew Levine, Long Beach, CA (US); Dimitrios Vatakis, Los Angeles, CA (US); Stefan Horvath, Los Angeles, CA (US); Dennis L. Kolson, Secane, PA (US)

(21) Appl. No.: 15/522,937

(22) PCT Filed: Oct. 30, 2015
(86) PCT No.: PCT/US2015/058343

§ 371 Date: Apr. 28, 2017

Related U.S. Application Data
(60) Provisional application No. 62/073,577, filed on Oct. 31, 2014.

(51) Int. Cl. A61K 31/575

(52) U.S. Cl. CPC A61K 31/575

(57) ABSTRACT

The present invention provides methods comprising treating and/or preventing cognitive dysfunction in subjects infected with HIV by administering an antioxidant inflammation modulator (AIM). The present invention also provides for methods for treating and/or preventing an HIV-associated neurocognitive disorder by contacting peripheral blood monocytes of an HIV+subject with an AIM.

### PRIORITY CLAIM

This application claims priority to U.S. Provisional Patent Application No. 62/073,577, filed on Oct. 31, 2014, which is hereby incorporated by reference in its entirety.

### GOVERNMENT SUPPORT

This invention was made with government support under R01-DA030913 awarded by the National Institutes of Health. The government has certain rights in the invention. This work was supported by the U.S. Department of Veterans Affairs, and the Federal Government has certain rights in the invention.

### BACKGROUND OF THE INVENTION

The Human Immunodeficiency Virus-1 (HIV) infects vital cells in the human immune system such as helper T cells (specifically CD4+ T monocytes, macrophages, and dendritic cells. HIV infection leads to low levels of CD4+ T cells through a number of mechanisms, including apoptosis of uninfected bystander cells, direct viral killing of infected cells, and killing of infected CD4+ T cells by CD8 cytotoxic lymphocytes that recognize infected cells. When CD4+ T cell numbers decline below a critical level, cell-mediated immunity is lost, and the body becomes progressively more susceptible to opportunistic infections. Advanced immunocompromised individuals can develop Acquired Immunodeficiency Syndrome (AIDS), a condition in which progressive failure of the immune system allows life-threatening opportunistic infections and cancers to thrive.

HIV enters the central nervous system as free virus or via infected cells, commonly such as monocytes, where it leads to neuropathological processes that can eventually result in neurocognitive and neuropsychiatric dysfunction. Common deficits include motor dysfunction, cognitive slowing, memory impairment, and behavioral change, which in turn may lead to impairments in day-to-day functioning. The range of neurocognitive and functional impairment cause by HIV is termed HIV-associated neurocognitive disorders (HAND). Currently, there is no known cure or preventative treatment for HAND. Considering that it occurs in an estimated 50% of infected of the over 1 million HIV+ individuals in the USA and 32 million worldwide, an effective prophylactic or treatment could have significant public health importance. Indeed, there is a long-felt need for pharmaceutical compositions and related methods for treating and/or preventing cognitive dysfunction in HIV-infected subjects.

### SUMMARY OF INVENTION

In one aspect, the invention relates to a method of treating or preventing cognitive dysfunction in a subject infected with human immunodeficiency virus (HIV), comprising administering to the subject in need thereof an antioxidant inflammation modulator (AIM). In certain embodiments, the subject has Acquired Immune Deficiency Syndrome (AIDS) and/or an HIV-associated neurocognitive disorder (HAND).

In another aspect, the invention relates to a method of treating or preventing HAND, comprising contacting peripheral blood mononuclear cells (PBMCs), such as monocytes, of an HIV+ subject with an AIM.

In certain embodiments, the AIM is a compound selected from:

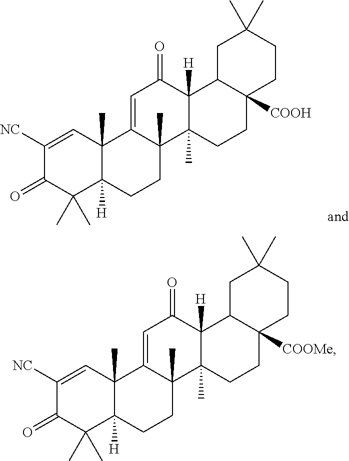

or a salt, ester, or prodrug thereof. In certain embodiments, the AIM is bardoxolone or bardoxolone methyl. In certain embodiments, the AIM is administered in a pharmaceutical composition with a pharmaceutically acceptable carrier.

### BRIEF DESCRIPTION OF THE DRAWINGS

FIG. 1 includes three panels identified as panels A, B, and C showing the results of preclinical testing of Bardoxolone in human monocyte-derived macrophages (MDM) and primary rodent neurons. Panel A shows hence-oxygenase-1 (HO-1) expression in HIV-infected MDM treated throughout the course of HIV infection with test drug (Bardoxolone or Tempol) or vehicle control. “Mock 1” and “Mock 2” correspond to supernatant from untreated, uninfected macrophages run in parallel with treated macrophages. “Vehicle 1” and “Vehicle 2” correspond to supernatant from macrophages that received 0.05% DMSO and no additional drug. Panel B shows the effects of Bardoxolone pre-treatment on reducing HIV replication in MDM, as demonstrated by reduced levels of HIV reverse transcriptase (RT) activity in the culture supernatants. This effect is seen at Bardoxolone concentrations as low as 5 nM. Panel C shows the results of a third experiment in which MDM culture supernatants are applied to cultured primary rodent neurons to test for MDM culture supernatant neurotoxicity.

FIG. 2 is a bar graph showing that Bardoxolone treatment resulted in reduced p24 gag expression, a measure of HIV replication, when HIV-infected UO1 monocytes were left untreated or were pretreated with Tempol or Bardoxolone and then exposed to lipopolysaccharide (LPS), an HIV transcription inducer.

### DETAILED DESCRIPTION

The present application discloses a use of antioxidant inflammation modulators (AIM) to treat or prevent cognitive dysfunction, preferably neurocognitive dysfunction, in a subject with human immunodeficiency virus (HIV).

In some embodiments, the subject has Acquired Immune Deficiency Syndrome (AIDS) and/or an HIV-associated neurocognitive disorder (HAND), HANDs are neurological disorders associated with HIV infection and AIDS.

In some embodiments, AIM is an oleanolic acid derivative, such as those described in U.S. Pat. Nos. 7,915,402, 8,071,632, 8,124,656, 8,440,820 and 8,440,854, hereby incorporated by reference in their entirety, and specifically with respect to the compounds disclosed therein. In certain embodiments, for example, the AIM is bardoxolone or bardoxolone methyl. In some embodiments, the AIM is a compound selected from

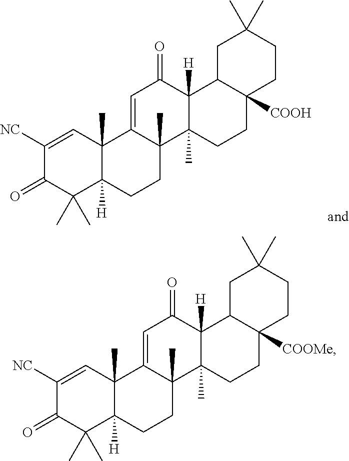

or a salt, ester, or

In certain embodiments, the AIM is a compound selected from the compounds as shown in Table 1 or a salt, ester, or prodrug thereof, such as those disclosed in U.S. Pat. No. 8,129,429 and International Publication No. WO 2013/163344, hereby incorporated by reference in their entirety and specifically with respect to the compounds disclosed therein.

TABLE 1

| Compound | Structure |
|---|---|
| Bardoxolone | 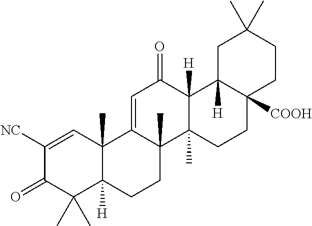 |
| Bardoxolone methyl | 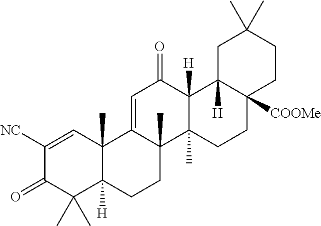 |
| AA | 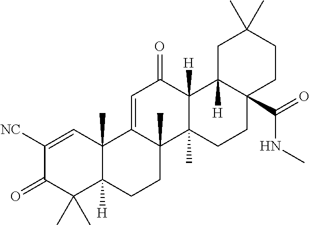 |
| AB | 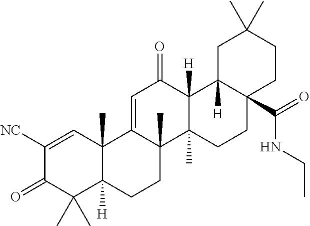 |
| AC | 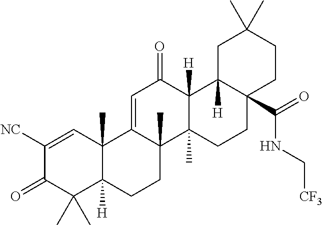 |
| AD | 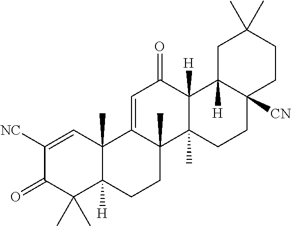 |
| AE | 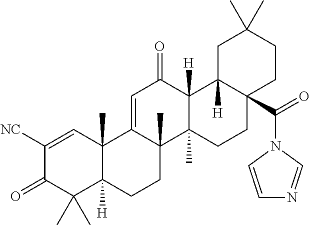 |
| AF | 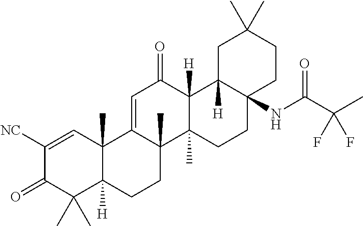 |
| AG | 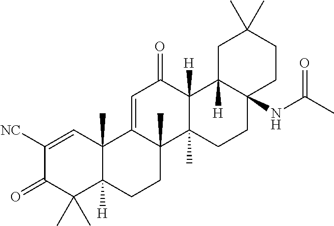 |
| AH | 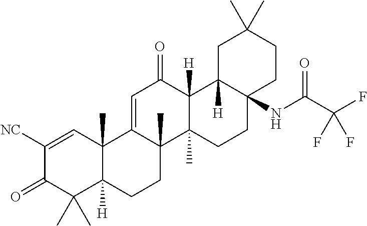 |
| AI | 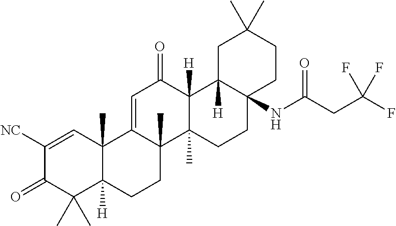 |
| AJ | 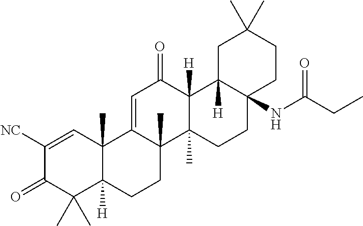 |
| AK | 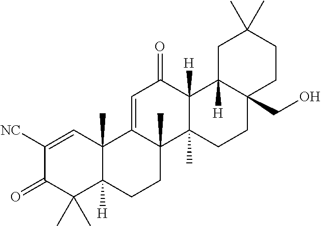 |

In certain embodiments, the invention relates to a method of treating or preventing a HAND, comprising contacting peripheral blood monocytes of an HIV+ subject with an AIM. Without wishing to be bound by theory, the presently disclosed methods relate in part to the identification of the relevance of the KEAP1/nrf2 pathway in a clinical cohort, and the observation that cells outside of the nervous system can be targeted to treat or prevent HAND.

### EXEMPLARY PHARMACEUTICAL COMPOSITIONS

In certain embodiments, the invention relates to a pharmaceutical composition comprising any one of the aforementioned compounds (e.g., bardoxolone, bardoxolone methyl) and a pharmaceutically acceptable carrier.

Patients, including but not limited to humans, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

The concentration of active compound in the drug composition will depend on absorption, inactivation and excretion rates of the drug as well as other factors known to those of skill in the art. It is to be noted that dosage values will also vary with the severity, of the condition to be alleviated, and possible drug-drug interactions with antiretroviral medications commonly taken by HIV+ patients. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

In certain embodiments, the mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition,

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose or sweetener as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antivirals, including but not limited to nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, carriers include physiological saline and phosphate buffered saline (PBS).

In certain embodiments, the active compounds are prepared with carriers that will protect the compound against rapid elimination from the body, such as a controlled release formulation, including but not limited to implants and microencapsulated delivery systems, such as those disclosed in International Publication No. WO 2010/093944, hereby incorporated by reference in its entirety, and specifically with respect to the formulations disclosed therein. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

The present invention also provides a pharmaceutical composition comprising the AIM compositions described herein. The pharmaceutical composition may also comprise suitable carriers or excipients.

The compositions and methods of the present invention may be utilized to treat a subject in need thereof In certain embodiments, the subject is a mammal such as a human, or a non-human mammal. When administered to an animal, such as a human, the composition is preferably administered as a pharmaceutical composition comprising, for example, a composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In preferred embodiments, when such pharmaceutical compositions are for human administration, e.g., for parenteral administration, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, powder, syrup, suppository, injection or the like. The composition can also be present in a transdermal delivery system; e.g., a skin patch. The composition can also be present in a solution suitable for topical administration, such as an eye drop.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

A pharmaceutically acceptable carrier can contain physiologically acceptable agents that act, for example, to stabilize or to increase the absorption of a compound of the invention (e.g., an AIM). Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention (e.g., an AIM). Liposomes, for example, which comprise phospholipids or other lipids, are nontoxic, physiologically acceptable and metabolizable carriers that are relatively simple to make and administer. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent or encapsulating Material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compositions may also be formulated for inhalation. In certain embodiments, a composition may be simply dissolved or suspended in sterile water. Details of appropriate routes of administration and compositions suitable for same can be found in, for example, U.S. Pat. Nos, 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In some embodiments of the present invention, the composition that is suitable for use in the invention may be administered orally, topically or parenterally, and in particular topically.

Dosage forms for the topical or transdermal administration include powders, sprays, ointments, pastes, creams, lotions, gels, solutions, patches and inhalants. The composition may be mixed under sterile conditions with a pharmaceutically acceptable carrier, and with any preservatives, buffers, or propellants that may be required.

The ointments, pastes, creams and gels may contain, in addition to an antibiotic, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the invention (e.g., an AIM), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The composition of the invention may be formulated with an excipient and component that is common for such oral compositions or food supplements, e.g., especially fatty and/or aqueous components, humectants, thickeners, preserving agents, texturizers, flavor enhancers and/or coating agents, antioxidants and preserving agents. Formulating agents and excipients for oral compositions, and especially for food supplements, are known in this field and will not be the subject of a detailed description herein.

In the case of a composition in accordance with the invention for oral administration, the use of an ingestible support is preferred. The ingestible support may be of diverse nature according to the type of composition under consideration. Tablets, gel capsules or lozenges, suspensions, oral supplements in dry form and oral supplements in liquid form are especially suitable for use as food supports.

Formulation of the oral compositions according to the invention may be performed via any usual process known to those skilled in the art for producing drinkable solutions, sugar-coated tablets, gel capsules, gels, emulsions, tablets to be swallowed or chewed, wafer capsules, especially soft or hard wafer capsules, granules to be dissolved, syrups, solid or liquid foods, and hydrogels allowing controlled release. Formulation of the oral compositions according to the invention may be incorporated into any form of food supplement or enriched food, for example food bars, or compacted or loose powders. The powders may be diluted with water, with soda, with dairy products or soybean derivatives, or may be incorporated into food bars.

In some embodiments, the composition according to the invention administered orally may be formulated in the form of sugar-coated tablets, gel capsules, gels, emulsions, tablets, wafer capsules, hydrogels, food bars, compacted or loose powders, liquid, suspensions or solutions, confectioneries, fermented milks, fermented cheeses, chewing gum, toothpaste or spray solutions.

An effective amount of the composition may be administered in a single dose per day or in fractional doses over the day, for example two to three times a day. By way of example, the administration of a composition according to the invention may be performed at a rate, for example, of 3 times a day or more, generally over a prolonged period of at least a week, 2 weeks, 3 weeks. 4 weeks, or even 4 to 15 weeks, optionally comprising one or more periods of stoppage or being repeated after a period of stoppage.

In certain embodiments, an AIM may be administered at a dose between 1 mg and 1,500 mg per day, such as between 5 mg and 1,300 mg per day, such as between 10 mg and 900 mg per day, such as between 20 mg and 600 mg per day, such as between 40 mg and 300 mg per day, such as between 150 mg and 350 mg per day, such as between 40 and 150 mg per day, such as between 25 mg and 150 mg per day such as between 2.5 mg and 150 mg per day, such as between 20 mg and 80 mg per day, or such as between 1 mg and 30 mg per day. In certain embodiments, an AIM may be administered at a dose of 1,300 mg/day, 900 mg/day, 600 mg/day, 350 mg/day, 300 mg/day, 250mg/day, 200 mg/day, 150 mg/day, 80 mg/day, 75 mg/day, 60 mg/day, 40 mg/day, 30 mg/day, 20 mg/day, 15 mg/day, 10 mg/day, 5 mg/day, or 2.5 mg/day. See Hong et al., Clin. Cancer Res.; 18(12):3396-3406, herein incorporated by reference in its entirety and specifically with respect to the doses.

This invention includes the use of pharmaceutically acceptable salts of compounds of the invention in the compositions and methods of the present invention. The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfornic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of a compound (e.g., an AIM). As another example, the salt may comprise less than one inorganic or organic acid molecule per molecule of base, such as two molecules of a compound an AIM) per molecule of tartaric acid.

In further embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol., diethanolamine, diethylamine, 2-(diethylamino)ethanol, ethanolamine, ethylenediamine, N-methylglucamine, hydrabamine, 1H-imidazole, lithium., L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

As one of skill in the art will appreciate, compositions of the present invention., not having adverse effects upon administration to a subject, may be administered daily to the subject.

Preferred embodiments of this invention are described herein. Of course, variations, changes, modifications and substitution of equivalents of those preferred embodiments will become apparent to those of ordinary skill in the art upon reading the foregoing description. The inventors expect skilled artisans to employ such variations, changes, modifications and substitution of equivalents as appropriate, and the inventors intend for the invention to be practiced otherwise than specifically described herein. Those of skill in the art will readily recognize a variety of non-critical parameters that could be changed, altered or modified to yield essentially similar results. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

### Definitions

For purposes of the present invention, the lowing definitions will be used (unless expressly stated otherwise):

As used herein, the term “administering” means the actual physical introduction of a composition into or onto (as appropriate) a subject. Any and all methods of introducing the composition into subject are contemplated according to the invention; the method is not dependent on any particular means of introduction and is not to be so construed. Means of introduction are well-known to those skilled in the art, and also are exemplified herein.

As used herein, the terms “effective amount”, “effective dose”, “sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition and provide either a subjective relief of a symptom(s) or an objectively identifiable improvement as noted by a clinician or other qualified observer. Amelioration of a symptom of a particular condition by administration of a pharmaceutical composition described herein refers to any lessening, whether permanent or temporary, lasting, or transitory, that can be associated with the administration of the pharmaceutical composition.

As used herein, the term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds in a formulation represented above can be replaced with the corresponding suitable prodrug, e.g., wherein a hydroxyl in the parent compound is presented as an ester or a carbonate or carboxylic acid present in the parent compound is presented as an ester.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, a “subject” means a human or animal (in the case of an animal, more typically a mammal). In one aspect, the subject is a human.

As used herein. “cognitive dysfunction” refers to a dysfunction in the ability of a subject to perceive, reason, or remember, or in the ability to acquire knowledge. Such symptoms of cognitive dysfunction may include, but are not limited to progressive loss of memory, cognition, reasoning, judgment, aspects of higher cortical function, diminished initiative, excessive distraction, speech, motor activity, recognition of perceptions, exaggerated or caricatured personality traits, irritability, excessive anger, violence, uncontrollable agitation, and delusions. Cognitive dysfunction includes but is not limited to amnesia, dementia, delirium, learning dysfunction, attention deficit disorders, speech dysfunction, speech deficits, learning disabilities, impaired communication skills, short term memory dysfunction, spatial learning dysfunction, and HAND. Such dysfunction may be associated with AIDS and other central nervous system conditions which cause such dysfunction. HAND ranges in severity from mild deficits that do not affect day-to-day functioning to frank dementia leaving the subject completely disabled and may include neurological disorders of various severity such as AIDS dementia complex (ADC) also known as HIV dementia and HIV-associated dementia (HAD), HIV encephalopathy, Mild Neurocognitive Disorder (MND), and Asymptomatic Neurocognitive Impairment (ANI). Features of HAND may include disabling cognitive impairment accompanied by motor dysfunction, speech problems and behavioral change.

The term “treating” is art-recognized and includes administration to the host of one or more of the subject compositions, e.g., to diminish, ameliorate, or stabilize the existing unwanted condition or side effects thereof.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### The importance of the KEAP1/nrf2 Pathway in a Clinical HIV+ Sample

Large scale gene expression and genomic studies were under taken in order to discovery pathways and biomarkers related to HAND. See also Levine et al., J. Neuroimmunology; 265:96-405 (2013), hereby incorporated by reference in its entirety. Transcriptome studies of HIV+ adults indicate that oxidative stress indicators within peripheral blood monocytes may be one of the strongest correlates of neurocognitive dysfunction in HIV° adults. This was shown by the increased expression of the Kelch-like erythroid CNC homologue (ECH)-associated protein 1 (KEAN.). Under normal physiological conditions, KEAP1 binds the nuclear transcription factor erythroid 2p45-related factor 2 (nrf2) in the cytoplasm. Exposure to oxidative stress elements (e.g., LPS, ROS, inflammatory cytokines, NOX-1) reduces the affinity between KEAP1 and nrf2, with the latter then being freed for nuclear translocation, where it binds to the antioxidant-response element (ARE) in the promoter region of genes encoding a myriad of antioxidant enzymes. Bcl-2, an antiapoptotic agent, is also a component of this pathway, as it induces nrf-2 nuclear translocation. This pathway is recognized as a target for neuroprotection, chemoprevention and chemoprotection, and anti-aging. It has also been indirectly implicated as a target for HAND, as among those molecules that inhibits the release of nrf2 is GSK3-beta, and GSK3-beta inhibitors have been shown to improve neurocognitive functioning in those with HIV. There is also evidence that modification of KEAP1 functioning influences transcriptional activity of NF-κβ, the most potent inducer of HIV-1 replication and also an inducer of inflammatory factors. Targeting NF-κβ and nrf2 via this pathway can both suppress pathological over-activation of NF-κβ signaling and activate protective nrf2, highly relevant tactics for HAND prevention.

The relevance of the KEAP1/nrf2 pathway to HAND has been directly demonstrated in vitro previously. See also Vijaya et al.., Neurochem Res. 37:1697-1706 (2012), hereby incorporated by reference in its entirety. For example, Gp-120 has been shown to upregulate nr12 expression in human astrocytes, and that this in turn stimulates gene and protein expression of the anti-oxidants HO-1 and Nq01. Expression of proinflammatory factors TNF-α, NF-κβ, and mellatoproteinase-9 are significantly elevated in astrocytes in which nrf2 expression is suppressed. Treatment of astrocytes with monosodium luminol upregulates nrf2 protein expression, and reduces oxidative damage. In addition, dietary supplements such as curcumin have been suggested as protective factors against HAND and other neurodegenerative diseases due to their influence on nrf2, Additionally, the nrf2 pathway was found to be suppressed in HIV-1 transgenic rats, as demonstrated both by decreased nrf2 protein expression and by heme-oxygenase-1 protein expression. HO-1 is a protective factor relevant to HAND, as described further in Example 3 below. See also Davinelli et al., Biogerontology, 15:449-461 (2014), hereby incorporated by reference in its entirety.

### Example 2

### The Identification of AIMs to Target the KEAP1/nrf2 Pathway in a Clinical HIV+ Sample

In addition to these compelling in vitro and in vivo findings, certain aspects of this invention demonstrate the relevance of the KEAP1/nrf2 pathway in a clinical HIV+ sample, and that cells outside of the nervous system can be targeted. This is highly important, as thus far all putative HAND treatments focusing on the CNS have been disappointing. A class of compounds, called antioxidant inflammation modulators (AIMs) has been identified with great promise in specifically targeting this pathway in peripheral blood mononuclear cells.

While other treatments have targeted other aspects of the cellular anti-oxidant response in brain cells, certain aspects of this invention discloses a crucial step that has a wide range of beneficial downstream effects, and it occurs in peripheral blood cells.

### Example 3

### Bardoxolone, an Exemplary AIM, Suppresses Viral Replication and is Neuroprotective

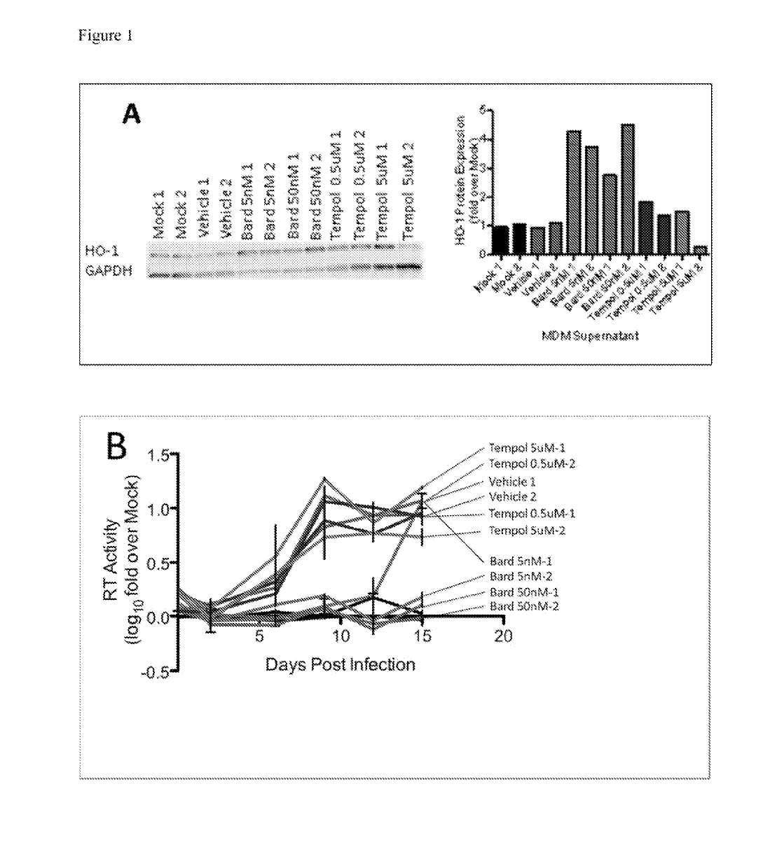

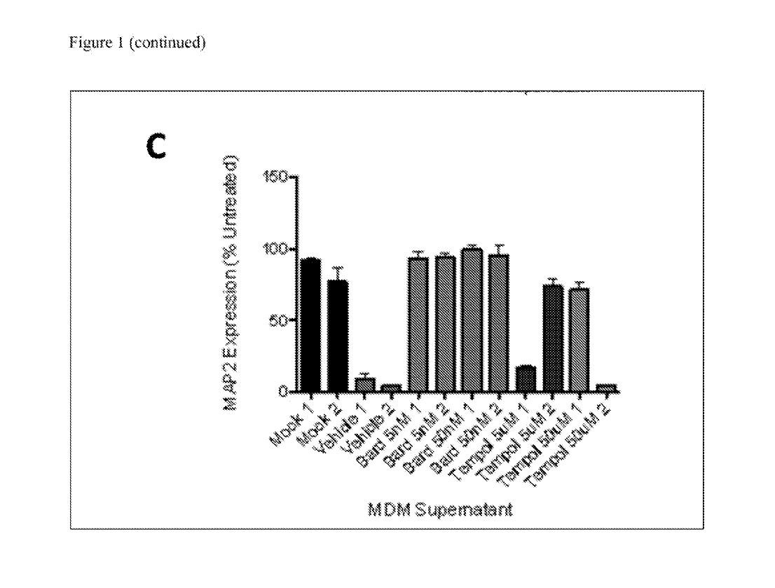

Monocyte-derived macrophages (MDM), obtained from uninfected donors, were pretreated with Bardoxolone (5 nM or 50 nM), Tempol, 4-hydroxy-2,2,6,6-tetramethylpiperidin-1-oxyl, (500 nM or 5 μM) or control (0.05% DMSO) for 24 hours prior to HIV infection. The respective drug or control was replaced with each medium exchange (every three days) throughout the course of infection, Bardoxolone treatment resulted in enhanced expression of HO-1 in HIV-infected MDM, as well as in non-infected MDM. See FIG. 1, Panel A. This enhanced HO-1 expression decreases release of soluble neurotoxins from infected MDM. “Mock 1” and “Mock 2” correspond to supernatant from untreated, uninfected macrophage run in parallel with treated macrophages. “Vehicle 1” and “Vehicle 2” correspond to supernatant from macrophages that received 0.05% DMSO and no additional drug. Final concentration of DMSO in all treated wells is 0.05%.

Viral replication was determined via reverse, transcriptase (RT) expression in the cell culture supernatant. The RT assay measured incorporation of ³²P dT into long chain poly rA. The effects of Bardoxolone pre-treatment on reducing HIV replication in MDM, as demonstrated by reduced levels of HIV reverse transcriptase (RT) activity in the culture supernatants are shown in FIG. 1, Panel B. This effect is seen at :Bardoxolone concentrations as low as 5 nM.

In addition, pre-treated neurons were exposed to the supernatants. Briefly, MDM culture supernatants are applied to cultured primary rodent neurons to test for MDM culture supernatant neurotoxicity. Loss of expression of microtubule associated protein-2 (MAP-2) in neurons is a sensitive marker of neuronal injury and death, Bardoxolone treatment of HIV infected MDM dramatically reduced the neurotoxicity of the MDM cell culture supernatants, as indicated by the maintenance or normal levels of MAP-2 in the neuron cultures, as compared to the vehicle control (non Bardoxolone) and to Tempol. This is an indication of neuronal health. See FIG. 1, Panel C.

### Example 4

### Bardoxolone Reduces Viral Expression

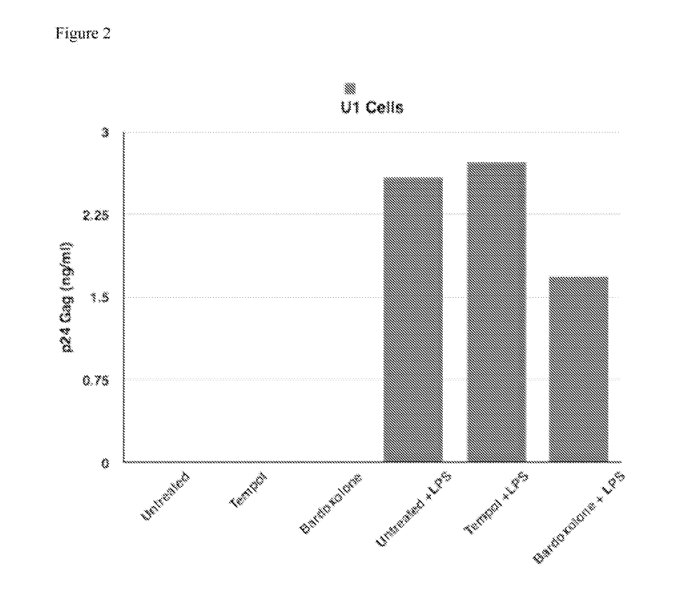

Evidence suggests that even low levels of viral expression in the CNS can lead to neuropathogenesis of HAND. As such, methods for reducing viral replication may reduce risk for HAND. In another series of experiments, HIV-infected monocytes were exposed to a virus replication stimulus to determine if pretreatment with Bardoxolone inhibited viral replication. The U1 monocytes (an HIV-infected monocytic line) do not typically express HIV until stimulated by external factors (e.g., LPS or proinflammatory cytokines). Here, a comparison of control cells with those pre-treated with either Tempol (1 μM) or Bardoxolone (250 nM) overnight was performed. The cells were then split into two conditions: LPS activated and non-LPS. As shown in FIG. 2, Bardoxolone pre-treatment decreased the amount of viral expression (p24 gag) following stimulation.

### REFERENCES

The following references, to the extent that they provide exemplary procedural or other details supplementary to those set forth herein, are specifically incorporated herein by reference.

Davinelli et al., Biogerontology, “Altered expression pattern of Nrf2/HO-1 axis during accelerated-senescence in HIV-1 transgenic rat,” 15:449-461 (2014)

Dohare et al., “The neuroprotective properties of the superoxide dismutase mimetic tempol correlate with its ability to reduce pathological glutamate release in a rodent model of stroke,” Free Radic. Biol. Med. 77:168-82 (2014).

Gonzalez et al., “The effects of tempol on cyclophosphamide-induced oxidative stress in rat micturition reflexes,” The Scientific World Journal. 2015:545048 (2015).

Hong et al., “A Phase I First-in-Human Trial of Bardoxolone Methyl in Patients with Advanced Solid Tumors and Lymphomas,” Clin. Cancer Res, 18(12) 3396-3406 (2012).

Levine et al., “Transcriptome analysis of HIV-infected peripheral blood monocytes: Gene transcripts and networks associated with neurocognitive functioning,” J. Neuroimmunology; 265:96-105 (2013).

Vijaya et al, “inhibition of Nuclear Factor Erythroid 2-Related Factor 2 Exacerbates HIV-1 gp120-Induced Oxidative and Inflammatory Response: Role in HIV Associated Neurocognitive Disorder,” Neurochem. Res.; 37:1697-1706 (2012).

Wanyong et at., “Tempol alleviates intracerebral hemorrhage-induced brain injury possibly by attenuating nitrative stress,” Neuroreport. 26(14):842-9 (2015).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating or preventing cognitive dysfunction in a subject infected with human immunodeficiency virus (HIV), comprising administering to the subject in need thereof an antioxidant inflammation modulator (AIM).

2. The method of claim 1, wherein the subject has Acquired Immune Deficiency Syndrome (AIDS).

3. The method of claim 1, wherein the subject has an HIV-associated neurocognitive disorder (HAND).

4. The method of claim 1, wherein the AIM is a compound selected from: 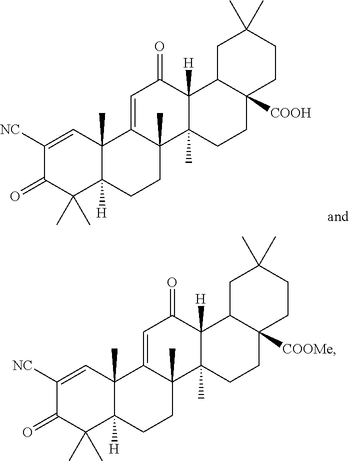
or a salt, ester, or prodrug thereof.

5. The method of claim 1, wherein the AIM is bardoxolone or bardoxolone methyl.

6. The method of claim 1, wherein the AIM is administered in a pharmaceutical composition with a pharmaceutically acceptable carrier.

7. A method of treating or preventing a HAND, comprising: contacting peripheral blood monocytes of an HIV+ subject with an AIM.

8. The method of claim 7, wherein the AIM is a compound selected from: 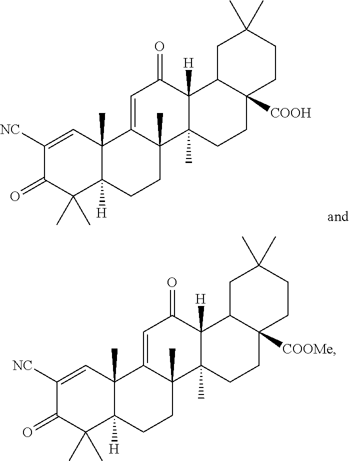
or a salt, ester, or prodrug thereof.

9. The method of claim 7, wherein the AIM is bardoxolone or bardoxolone methyl.
